(19) United States
(12) Patent Application Publication
(10) Pub. No.: US 2022/0145236 A1
(43) Pub. Date: May 12, 2022

(54) SPECIMEN COLLECTION FOR MICROBIAL BURDEN CLASSIFICATION AND SPECIMEN TRANSPORTATION TO LAB FOR REPORTING DIRECT-FROM-SPECIMEN ID AND AST

(71) Applicant: GeneFluidics, Inc., Irwindale, CA (US)

(72) Inventors: Vincent Gau, Irwindale, CA (US); Michael Tomasek, Huntington Beach, CA (US); Jade Chen, Diamond Bar, CA (US); Amelia Kung, Irwindale, CA (US)

(21) Appl. No.: 17/524,568

(22) Filed: Nov. 11, 2021

Related U.S. Application Data
(60) Provisional application No. 63/112,499, filed on Nov. 11, 2020.

(51) Int. Cl. C12N 1/04; C12M 1/00; C12Q 1/04; B65D 81/18; G01N 27/327

(52) U.S. Cl. CPC C12N 1/04; C12M 23/52; G01N 27/3275; B65D 81/18; G01N 27/3271; C12Q 1/04

(57) ABSTRACT

The disclosure relates to methods and systems for biological assays and assay transport systems.

### CROSS REFERENCE TO RELATED APPLICATIONS

The application claims priority to U.S. Provisional Application No. 63/112,499, filed Nov. 11, 2020, the disclosure of which is incorporated herein by reference.

### TECHNICAL FIELD

The disclosure relates to methods and systems for biological assays and assay transport systems.

### BACKGROUND

The clinical interpretation of urine and blood culture results, as well as the cost-effectiveness of urine and blood cultures, depends on many variables, the most important of which is the proportion of cultures that are contaminated by skin flora. Specimens should be collected in such a way that contamination by indigenous flora is minimized. This is of paramount importance for cultures of blood and urine or fluids in which infection is often caused by indigenous flora and for specimens collected from sites of putative infection that are contiguous to, or immediately adjacent to, cutaneous or mucosal surfaces.ⁱ Contaminations are attributed to the transfer of microorganisms from the immediate environment of the patient or, more rarely, from healthcare workers' hands. The predictive value of urine and blood culture time to positivity is based on the premises that the bacterial inoculum in a true bacteremia is higher than in a urine and blood culture contaminant's and grows faster. When a coagulase-negative staphylococci is isolated, a longer growth time is usually considered in favor of a contaminant, and growth times between contaminants and true pathogens overlap.^{ii, iii}

The specimens for microbiological testing must also be collected with use of strict aseptic technique from anatomic sites most likely to yield pathogenic microorganisms, therefore bacterial cultures are not presently ready for home tests with self-collected specimens. The blood volume needed for blood culture and the need of a phlebotomist also exclude the use of finger prick blood samples. Sufficient material must be submitted for cultures and other tests, and blood volume is crucial to ensure accuracy in blood cultures. With limitations on self-collection methods due to the required volume for blood cultures and concerns regarding contamination by skin flora for both urine and blood cultures, the presence of healthcare professionals at collection sites is essential to ensure accurate clinical interpretation of culture results. Thus, point-of-care home tests with self-collected urine or blood samples are currently not feasible. There is a need for better interim tests between clinical laboratory cultures in hospital settings and self-collection home tests to rapidly identify patients with resistant pathogens and for more judicious use of broad-spectrum antibiotics for empiric sepsis treatment, especially in out-of-hospital settings. The specimen collection and transportation protocols developed in this study aimed to utilize the specimen transportation time as part of the viability preservation for urine cultures and viability enhancement for blood cultures.

A recent study demonstrated that the measurement of 21 blood biomarkers from 134 blood samples taken by emergency medical services (EMS) and placed in a Coleman cooler box on the worktop inside the patient's cabin in an ambulance truck during the hospital transfer was exactly like the one immediately analyzed in the hospital setting.^{iv} The possible benefits to patient, outcome deriving from out-of-hospital blood sampling were limited in this study, because blood samples were taken by EMS, and the time saved is only 30 minutes or less on the ambulance ride to the emergency department in the hospital. However, in the context of nation-wide shipping, which averages 19 hours specifically for the overnight express option, incubation may be performed en route, allowing the incubation period to be fully completed during transportation and thus reducing the overall testing time, which for current ID assays would originally include a 2 and 5-hour viability culture for urine and blood, respectively. Extended growth time often causes contaminants in urine, which would have otherwise remained below the clinical cutoff, to exceed the bacterial inoculum of the true infection. Therefore, the type of tubing as well as the thermal conditions of the packaging are both crucial to the regulation of such inaccuracies.

### SUMMARY

The disclosure demonstrate an evidence-based specimen transportation pack design considering the effects of weather conditions and transportation time on the growth of contaminants and bacteremia to further rapid direct-from-specimen antimicrobial susceptibility test (AST), and pre-hospital molecular phenotyping ID/AST diagnostics.

The disclosure provides a method to collect, pack and transport a specimens for microbiological testing, which comprises, inoculating the specimen in a plurality of wells, wherein each well of the plurality of wells comprises a control, different dilutions and/or different antimicrobial agents; determining a change of one or more growth markers to assess a microbial burden or an antimicrobial susceptibility from an identified or an unidentified pathogen(s) under various antimicrobial exposure conditions compared to a Growth Control (GC) condition without any antimicrobials, wherein the microbial burden is determined by a change of the one or more growth markers from unidentified pathogen growth within a pre-determined viability culture time as part of the specimen transportation time; and/or wherein the antimicrobial susceptibility is determined by a change of one or more growth markers within an antibiotic exposure time as part of the specimen transportation time from unidentified pathogen diluted at different dilution levels with various drug and/or pathogen ratios compared to a Growth Control (GC) condition without any antimicrobials and/or wherein the antimicrobial susceptibility is determined by a change of one or more growth marker from identified pathogen with various drug and/or pathogen ratios compared to Growth Control (GC) without any antimicrobials, and/or wherein the microbial burden or antimicrobial susceptibility are determined by pathogen growth within a pre-determined viability culture time after removing a matrix interference components, and/or wherein the microbial burden or antimicrobial susceptibility are determined by pathogen growth within a pre-determined viability culture time after concentrating the pathogens in the raw specimens. In one embodiment, the one or more growth markers comprises nucleic acids, proteins, phenotypic characteristics, and/or visual observation. In a further embodiment, the one or more growth markers is RNA and the change of RNA content is quantified with a molecular analysis assay. In still a further embodiment, the molecular analysis assay is selected from the group consisting of species-specific quantification, group-specific quantification, and universal quantification. In another embodiment, the identified or unidentified pathogen is selected from the group consisting of E. coli, Klebsiella pneumoniae, and methicillin-resistant Staphylococcus aureus (MRSA). In yet another embodiment, the identified or unidentified pathogen are selected from Enterobacteriaceae, Gram-negative and Gram-positive bacteria. In a further embodiment, the growth marker is RNA and the change of RNA content is quantified with molecular analysis assays with enzymatic signal amplification with electrochemical sensors. In another embodiment, microbial growth comprises a growth condition in microdilution, macrodilution, agar plating, growth media culture, growth in clinical specimens or processed specimens. In a further embodiment, the growth conditions comprise temperature control, a preservative, breakage prevention, leakage prevention, and differential viability culture time to distinguish contaminants. In yet another embodiment, differential viability culture time to distinguish contaminants comprises a culture time needed at around the limit of detection. In another embodiment, the antimicrobial exposure conditions comprise microdilution, macrodilution, agar plating, growth media culture, or growth in clinical specimens or processed specimens with antimicrobial conditions. In a further embodiment, antimicrobial conditions comprise a set number of antimicrobial concentrations, a range of antimicrobial concentrations, various drug-to-microbe ratios, and/or different antimicrobial exposure times. In yet a further embodiment, the set number of antimicrobial concentrations is comprised of susceptible, intermediate and/or resistant breakpoints. In another embodiment, the set number of antimicrobial concentrations is comprised of 2-fold increase or decrease from a susceptible, intermediate and/or resistant breakpoints. In yet another embodiment, the set number of antimicrobial concentrations is comprised of more than 2-fold increases or decreases from a susceptible, intermediate and/or resistant breakpoints. In still another embodiment, the set number of antimicrobial concentrations comprises 2 to 12 antimicrobial conditions. In another embodiment, the different dilution levels are comprised of a set number of dilution levels from a raw specimen, a range of dilution levels from a raw specimen, and different levels of pathogen concentrating step. In a further embodiment, the different dilution levels are selected from 1×, 0.5×, 0.3×, 0.1×, 0.01×0.001×0.0001× and/or 0.00001×. In another embodiment, the method further comprises removal of supernatant from the specimen and before determining the microbial burden or antimicrobial susceptibility after a centrifugation step. In a further embodiment, the centrifugation step can include a specimen pre-conditioning step comprised of red blood cell lysis or thinning agent to reduce viscosity. In another embodiment, the centrifugation step can comprise centrifugation for about 5 min, 10 min, 20 min or more at about 0.1 G, 0.5 G, 1 G, 2 G or more and/or a pellet volume of about 100 μL, 150 μL, 200 μL or more. In another embodiment, the temperature control is accomplished by using one or more cold packs, one or more heat packs, use of a temperature-controlled device, or use of a thermal isolated device. In another embodiment, the preservative comprises boric acid or a composition that inhibits growth of a pathogen or cells. In another embodiment, the breakage prevention is accomplished by the addition of an outer packaging box made of sturdy material for protection, such as plastic, wood, and/or metal. In another embodiment, the leakage prevention is accomplished by the use of one or more of adding an absorbent material, adding a sealing bag, and/or adding a sealing tape to each specimen collection tube or container. In another embodiment, the differential viability culture comprises a set viability culture time with preservative and temperature control, a set viability culture time with temperature control but without preservative, and any combination of the use of culture time, temperature control and preservatives. In another embodiment, the pre-determined viability culture time as part of specimen transportation time is selected from 5 min, 30 min, 1-hour, 2-hours, 3-hours, 6-hours, 12-hours, 18-hours and 24-hours. In yet another embodiment, the various antimicrobials comprise one, three, five, or ten or more antimicrobials. In another embodiment, the antimicrobial susceptibility testing comprises the susceptibility of a pathogen in a monomicrobial specimen, the susceptibility of pathogens in a polymicrobial specimen, the susceptibility of a multiple-drug resistant pathogen in a monomicrobial infection, and the susceptibility of each multiple-drug-resistant pathogen in a polymicrobial infection. In another embodiment, the set number of antimicrobial concentrations comprises 13-96, 96-256, 256-1024 singular antimicrobial conditions to assess antimicrobial susceptibility profiles comprising the susceptibility of each pathogen in a polymicrobial specimen, the susceptibility of a multiple-drug-resistant pathogen in a monomicrobial infection, and the susceptibility of each multiple-drug-resistant pathogen in a polymicrobial specimen. In another embodiment, the set number of antimicrobial concentrations comprises 13-96, 96-256, 256-1024 singular and/or combinational antimicrobial conditions to assess antimicrobial susceptibility profiles comprising the susceptibility of each pathogen in a polymicrobial specimen, the susceptibility of a multiple-drug-resistant pathogen in a monomicrobial infection, and the susceptibility of each multiple-drug-resistant pathogen in a polymicrobial specimen. In a further embodiment, the antimicrobial susceptibility profile comprise no observed growth, limited growth, minimum growth, and/or relatively low growth. In another embodiment, the antimicrobial susceptibility profile is performed on a homogeneous microbial population, a heterogeneous microbial population, a pseudo-homogeneous microbial population and/or a pseudo-heterogeneous microbial population. In a further embodiment, the antimicrobial susceptibility profile is performed on a homogeneous resistant microbial population, a heterogeneous resistant microbial population, a pseudo-homogeneous resistant microbial population and/or a pseudo-heterogeneous resistant microbial population. In a further embodiment, a majority the population of the heterogeneous microbial population is susceptible and a minority of the population is resistant. In another embodiment, the pre-determined viability culture time comprises 2-hours, 3-hours, 6-hours, 12-hours and 18-hours in order to observe the growth of a minority population after the inhibited growth of a majority susceptible population. In another embodiment, a clinical specimens is a swab or a bodily fluid selected from the group consisting of urine, blood, sputum, or surgical drain fluids.

### BRIEF DESCRIPTION OF THE FIGURES

FIG. 1A-B is an illustration of (a) Patient-side specimen collection and transportation package, (b) specimens shipped from NYPQ via FedEx Clinical Pak.

FIG. 2A-D shows tested urine on Day 0 (a) and Day 3, all stored in C&S tubes. Condition 1 (b): 4C storage, 1 hr return to RT before beginning assay with a 1 hr viability culture. Condition 2 (c): 4C storage, begin assay immediately with 1 or 2 hour culture. Condition 3 (d): RT storage, begin assay immediately with 1 or 2 hour culture. Proved that 4C storage or cold pack is needed.

FIG. 3A-D shows urine calibration curve tested at 0, 12, and 24 hrs. (a) and (b) urine stored with one cold pack and tested with either 1 or 2 hours of viability culture. (c) urine stored with one heat pack and cultured for one hour. (d) thermal tracking profile of transportation package with cold or heat packs.

FIG. 4A-C shows simulated urine specimen shipment using the transportation pack finalized from FIGS. 1 and 2.

FIG. 5A-B shows simulated enhanced viability recovery of contrived blood samples at 0.47, 4.7, and 47 CFU/mL tested every 2 hours after storage in a thermal bag with 2 heat packs. (a) all blood samples reported positive after 10 hours of simulated transportation time, (b) simulated overnight shipping with blood samples contrived at 0.83 and 5.3 CFU/mL.

FIG. 6A-B shows a feasibility study of specimen transportation of contrived (a) blood and (b) urine samples shipped from NYPQ to GeneFluidics through FedEx Clinical Pak overnight express.

FIG. 7 shows a comparison of susceptibility reporting timelines for gold standard and the proposed patient-side initiated system.

FIG. 8 shows total turnaround time (TAT) for ID and AST in clinical microbiology laboratories.

FIG. 9A-B shows Calibration curves of configurable ID protocols with various TAT and LODs. 2C Triple dynamic responses for ciprofloxacin AST.

FIG. 10A-E shows molecular microbial burden quantification of urine samples for the current UtiMax transportation protocol for up to 3 days (A) and the proposed nursing home transfer for one and two hours (B). The viability assessment will be completed in the BsiMax system in the clinical microbiology lab if the hospital transfer time is less than the required viability assessment time. Molecular microbial burden quantification (B) after 2, 4, 6, 8 and 10 hours of the transportation time from whole blood samples spiked at 0.5 to 50 CFU/mL to ensure full recovery and viability of blood borne pathogens. Proposed transportation pack (D) with the heat pack to be used.

FIG. 11 provides a simplified outline depicting the difference between conventional analysis and an analysis based upon the present disclosure.

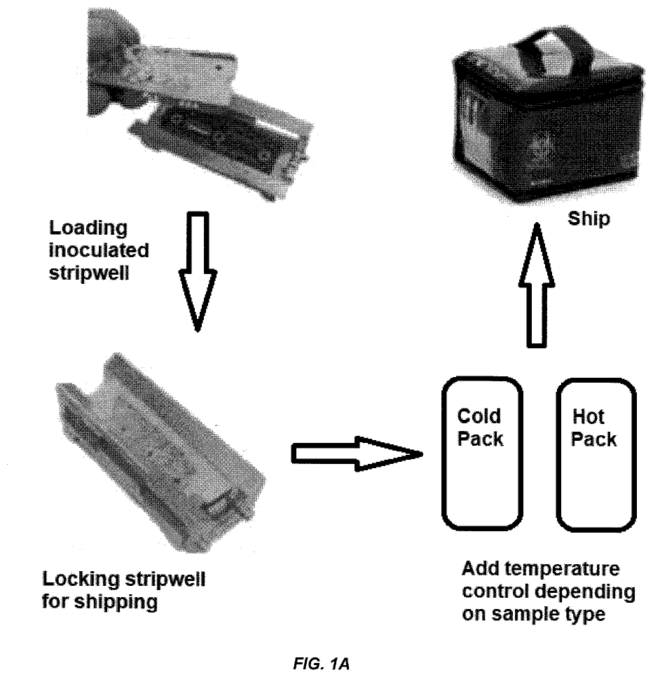

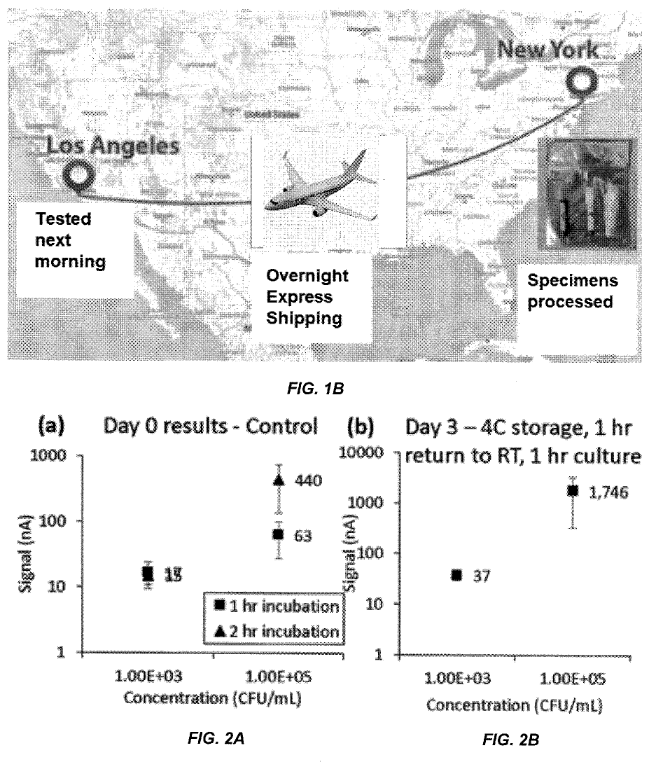

### DETAILED DESCRIPTION

As used herein and in the appended claims, the singular forms “a,” “an,” and “the” include plural referents unless the context clearly dictates otherwise. Thus, for example, reference to “a subject” includes a plurality of such subjects and reference to “the sample” includes reference to one or more samples and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7A11 headings and subheading provided herein are solely for ease of reading and should not be construed to limit the invention. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and specific examples are illustrative only and not intended to be limiting.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Sepsis is a major source of morbidity and mortality among the nation's estimated 1.4 million nursing home residents. In the emergency department, nursing home residents are 17 times more likely to be diagnosed with sepsis than non-nursing home residents, such that nearly 4% of emergency department visits among nursing home residents include a diagnosis of sepsis. Furthermore, when sepsis occurs, it is more likely to be severe if the patient is a nursing home resident, leading to higher rates of intensive care unit admission, longer hospital stays, and higher mortality rates when compared to non-nursing home residents. Moreover, older adults who survive sepsis are at increased risk of new or worsening cognitive impairment and functional decline. A 2019 sepsis study found that each additional hour from emergency room arrival to antibiotic administration increased odds of 1-year mortality by 10%. However, another 2020 large study found treatment with unnecessary broad empiric antibiotics was associated with a 22% increase in mortality. Among 17,430 culture-positive community-onset sepsis patients aged 57-81 in US hospitals, 67% received antibiotics targeting drug-resistant organisms like methicillin-resistant Staphylococcus aureus (MRSA) and Pseudomonas aeruginosa, yet resistant Gram-positive organisms were isolated in only 13.6% of patients and resistant Gram-negative organisms were isolated in just 13.2% of patients. Both under treatment (failure to cover organisms) and over treatment (resistant organisms targeted but not isolated) were associated with higher mortality after detailed risk adjustment. These findings underscore the need for better tests to rapidly identify patients with resistant pathogens and for more judicious use of broad-spectrum antibiotics for empiric sepsis treatment.

Problems with high infection rates among the elderly are exacerbated by a rapidly growing global elderly population. Elderly nursing home residents are highly susceptible to infections owing to the higher likelihood of compromised physiologic barriers, immunosuppression, malnutrition, dehydration, comorbidities, and functional impairments. In the US, over 4 million people reside in nursing homes and skilled nursing facilities, and 1 to 3 million serious infections occur each year, placing a significant burden on long-term care facilities. Given their congregate nature and resident population served (older adults often with underlying chronic medical conditions), nursing home populations are at high risk of contracting infections such as COVID-19 and sepsis caused by multi-drug-resistant organisms (MDROs) including carbapenemase-producing organisms and Candida auris. Bacterial infections are among the most common causes of morbidity and mortality in nursing homes and other long-term care facilities. MDROs represent an ever-increasing share of causative agents of infection, and their prevalence in nursing homes now matches or exceeds the prevalence in acute-care facilities. Despite the identification of nursing homes as a major reservoir of MDROs for the community at large, the regulations for staffing a part-time infection preventionist without a specific required time commitment at nursing homes are not sufficient to provide effective infection management.

Diagnosis of sepsis in older adults can be especially difficult and often goes under-diagnosed. Older adults with sepsis often present with atypical, nonspecific symptoms. Sepsis is typically diagnosed using the systemic inflammatory response syndrome (SIRS) criteria, but typical signs of sepsis and the SIRS criteria are not commonly seen in geriatric patients. The body's baseline temperature changes with age and can oftentimes be 0.6-0.8° C. lower in older adults than in younger adults when assessing the SIRS criteria for temperature (>38° or <36° C.). There is a decreased temperature response to infection as a result of decreased cytokine production, decreased sensitivity of the hypothalamus to cytokines, and poor peripheral thermoregulation. Malnutrition also leads to a decreased temperature response to infection. Older adults with urinary tract infections often present with confusion and they are less likely than younger adults to present with the classic symptoms of urinary frequency and pain. Moreover, older adults with pneumonia are more likely to present with generalized weakness, falls, and hypoxemia, rather than the typical symptoms of fever and chest pain. Biomarkers such as erythrocyte sedimentation rate, CRP, lactate, and procalcitonin are often used for diagnosis of host response to sepsis, but these markers may be unreliable indicators of infection due to an elevated baseline due to aging and the presence of multiple disease states. For example, the presence of altered mental status is a nonspecific marker of infection in older patients and does not necessarily indicate a nervous system infection as it would in younger adults.

Hospitals need to clear out patients who no longer need acute care, but nursing homes are hesitant to accept patients discharged from hospitals due to the fear they have been exposed to the coronavirus. In the US, more than one-third of older sepsis survivors are admitted to post-acute care facilities following hospital discharge for ongoing skilled nursing care and rehabilitation, such as physical, occupational, and speech therapy. However, numerous outbreaks of SARS-CoV-2 infection have occurred in skilled nursing facilities. Patients and families may therefore be reluctant to accept placement at a post-acute care facility, and even if willing, facility availability may be limited due to closures, downsizing, or additional placement requirements to mitigate the spread of COVID-19. Early etiological diagnosis and characterization of microbial susceptibility of the infection are becoming central in nursing home sepsis management. Still, limitations in conventional diagnosis and patient stratification contribute to the high mortality rates among septic patients, despite new antimicrobials and resuscitation agents. Nursing homes are equipped to provide care to ill residents, including treatment of pneumonia, urinary tract infections, skin infections, and fevers, but are not able to identify sources of infection and causative pathogens. Effective management of infections such as uncomplicated UTI in nursing homes can be assisted by patient-side molecular phenotyping diagnostics initiated in nursing homes to monitor an acute change in status event or aid in the general treatment of residents in-house.

There are significant risks and possible adverse consequences of prolonged inappropriate antibiotic therapy in the elderly, including risks of drug interactions, side effects related to age or disease-related changes in metabolisms, and risks associated with MDROs and Clostridium difficile. The 2018 Surviving Sepsis Campaign (SCC) guideline updates strongly recommend that the administration of intravenous broad-spectrum antibiotics should be initiated as soon as possible, preferably within an hour of sepsis recognition. However, the Infectious Diseases Society of America (IDSA) does not agree with “one size fits all” recommendations based upon varying definitions of sepsis that do not clearly differentiate between sepsis and septic shock stating that following these recommendations, while life-saving for those with shock, may lead to overtreatment with broad spectrum antibiotics for those with milder variants of sepsis. However, there are not any recommendations specifically designed for older adults. For the treatment of septic shock syndrome, limited literature exists to guide appropriate selection and dosing of pharmacotherapy in older adults.

At-home testing is cost-effective, rapid, and could assist in avoiding hospital visits during a pandemic. Such tests could reduce the risk of contracting or spreading a virus such as SARS-CoV-2. Despite the convenience and timeliness of direct-to-consumer tests, they present some significant risks that current technologies cannot fully address yet. Skin flora contamination and insufficient specimen volume are two major limitations preventing self-collection microbiological testing for home use.

The disclosure provides a hybrid testing procedure to bridge the laboratory test with patient-side specimen collection and transportation protocols for molecular microbial classification of causative bacterial infection and early identification of microbial susceptibility profiles directly from whole blood or urine specimens collected patient-side by health care workers such as a phlebotomist in a nursing home or family clinic. The disclosure demonstrates that using various transportation conditions (tube types, temperature, duration, inclusion of boric acid), for direct-from-urine ID, the viable pathogen at the clinical cutoff of 10⁴ CFU/mL was detected with species-specific molecular assays while contaminants (skin flora at concentrations <10⁴ CFU/mL) were not reported positive. For direct-from-blood ID assays, contrived blood samples at as low as 0.8 CFU/mL can be reported positive after specimen transportation without the need of blood culture.

Specimens submitted for microbiological testing require proper handling from the time of collection through all stages of transport, storage, and processing, but these three stages have never been utilized as part of a test to report pathogen identification results within one day (i.e., in other words the testing began following transport). The patient-side specimen collection and transportation protocol of the disclosure can report contrived blood sample positive at concentrations as low as 0.83 CFU/mL upon receiving the specimen shipment (e.g., receiving a FedEx Clinical Pak shipment) at the testing site (e.g., as exemplified in a New York to Los Angeles shipment). The urine collection and transportation protocol can report uropathogens contrived at >10⁴ CFU/mL positive while reporting concentrations ≤10⁴ CFU/mL, common of contaminants, as negative.

The protocol optimization goals are different for whole blood and urine. In some embodiments, heat packs were added to the blood transportation pack to keep bloodborne pathogens in the log phase and enhance the viability growth during the transportation period. In other embodiments, cold packs were added for urine transportation to avoid bacterial overgrowth causing false positives with skin flora contamination. The number of cold or heat packs added to the package can change the thermal profile, and the disclosure provides an optimal condition for both blood and urine transportation packaging.

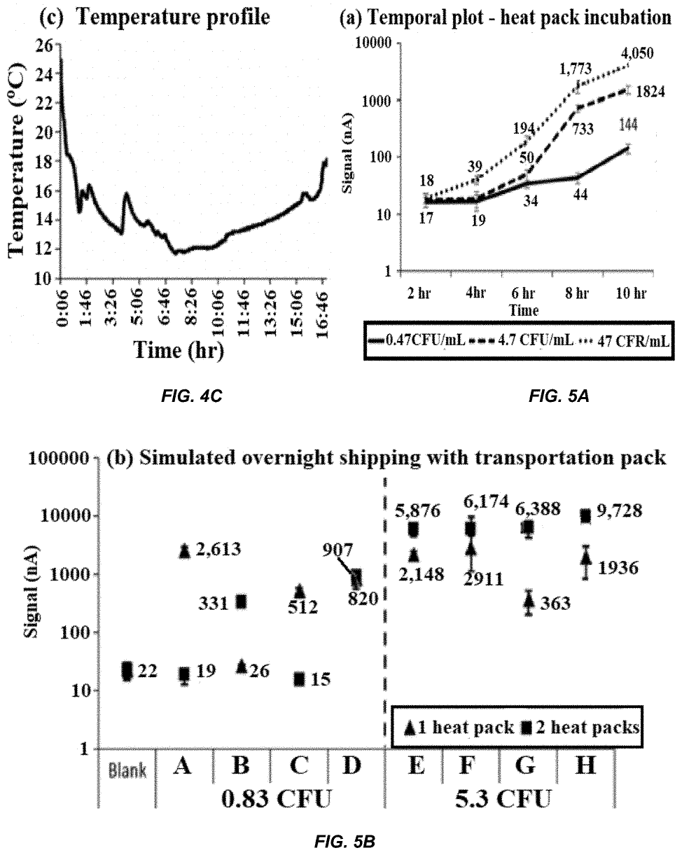

To avoid false positives from skin flora, boric acid is routinely used to preserve the viable colonies. However, a drop in signal level was observed from the molecular analysis assay quantifying the 16s rRNA content of viable pathogens. The presence of boric acid and long transportation time could put the uropathogens into stationary mode resulting in lower RNA content caused by decreased colony count over time. This was addressed by adding viability culture time as part of the automated molecular analysis assays as shown in FIG. 4b. The closed-loop controlled thermal profile inside the system provided a consistent environment to bring the uropathogens back to log phase. The detection sensitivity was set to meet the clinical cutoff of 10⁴ CFU/mL, and the total assay time can be adjusted to achieve different levels of limit of detection by varying the viability culture time inside the system.

One focus of the blood collection and transportation protocol development falls on the ability to detect low abundancy (<1 CFU/mL) of pathogens in whole blood samples. Therefore, the primary goal is to prevent the loss of pathogen due to extreme conditions, and the secondary goal is to enhance the viable colony count during transportation. FIG. 5a suggests that the transportation time needs to be longer than 6 hours in order to call 5 and 50 CFU/mL positive, and 10 hours for 0.5 CFU/mL. If the actual transportation time is less than the required time, the total assay time with the automated system at the receiving end will be adjusted based on the sample scan time stamp. No false positives were observed from all negative blood samples. The current blood specimen processing protocol is part of the automated procedure done by a robotic system. A separate specimen processing unit can be implemented at the patient site in order to automate this procedure to avoid specimen contamination and reduce the burden of healthcare professionals.

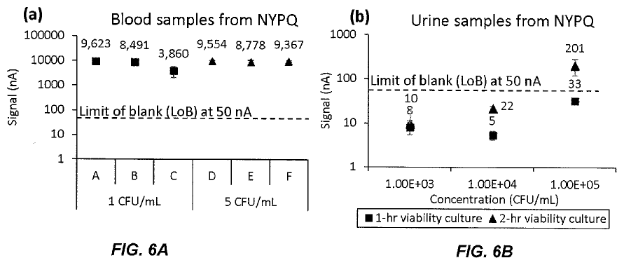

FIG. 6 provides a pilot feasibility transportation result. NYPQ staff at the clinical microbiology lab followed the work instruction to contrive, pack, and ship urine and blood samples via a FedEx Clinical Pak to GeneFluidics, Los Angeles, Calif. All samples were tested upon receiving, and results agreed with the simulated studies as shown in FIGS. 4 and 5.

Timely tracking of infections and other causative-pathogen-specific events can facilitate antibiotic stewardship program quality improvement and monitoring of the effectiveness of infection management measures. The disclosure provides a protocol to leverage the outcome of the current molecular analysis platform and establish a patient-side-initiated diagnostics platform to assess multivariable factors such as species and phenotypes of causative pathogens and microbiological response to antibiotics. Success in such treatments is the consequence of a multitude of factors, including pharmacokinetics, effective in vivo drug concentrations, microbial species and host interactions.

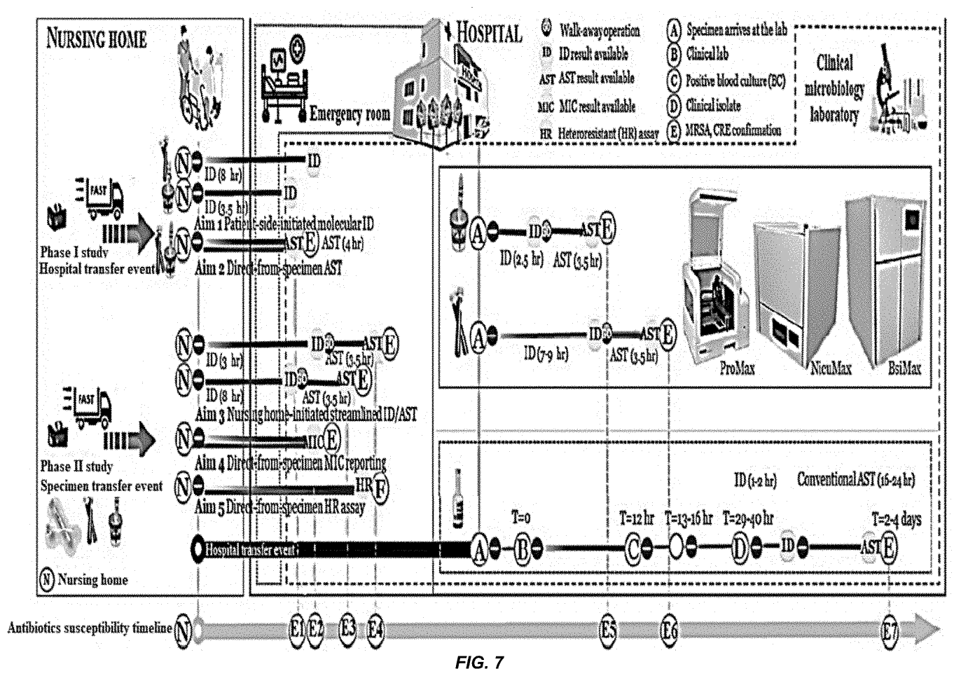

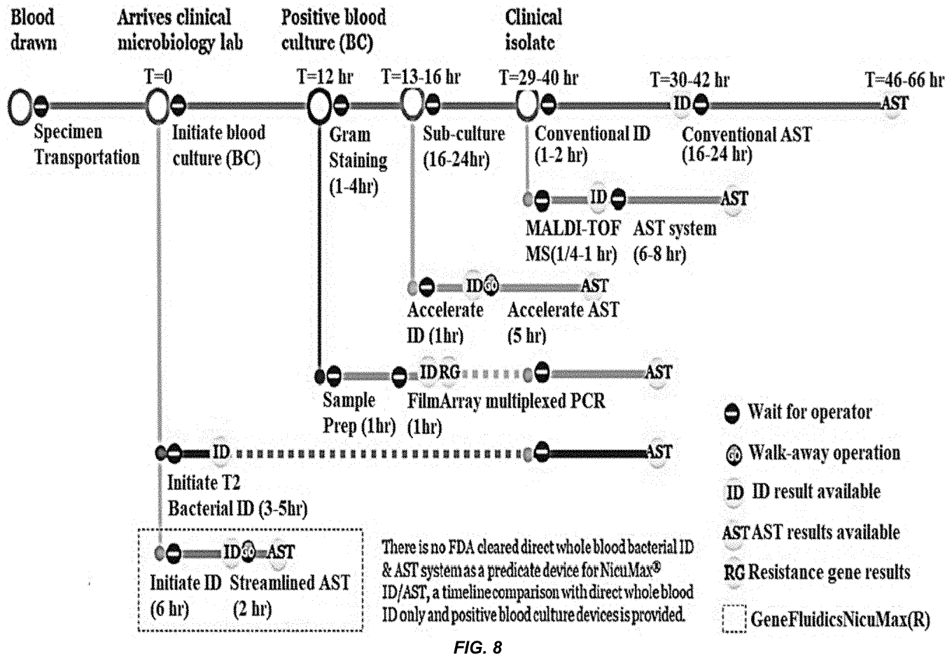

A comparison of GeneFluidics' direct-from-specimen streamlined ID/AST system (upper right blue box in FIG. 7) with FDA-cleared systems is illustrated in FIG. 8. FIG. 7 shows a comparison of timelines for the gold standard blood culture method (lower right grey dashed box), the current streamlined ID/AST system (ProMax, NicuMax and BsiMax in blue box), and the proposed patient-side initiated system (green boxes initiated in nursing homes through the blue dotted box of the emergency department (ED) and completed in the blue dashed box of the clinical microbiology lab in the hospital). There is a clear need for a method that significantly reduces the total assay time (TAT) to report antimicrobial susceptibility responses of causative pathogens to antibiotic regimen options to help physicians make informed decisions before the second administration of empirical antibiotics. Nursing home residents are initially considered as outpatients after being transferred to the emergency room of a hospital (bold black line at the bottom). A hospital outpatient-based study in the US evaluating the appropriateness of IV antibiotics prescribed found that one-third of antibiotic doses were inappropriate. The conventional blood culture and the current streamlined ID/AST start after the blood sample of nursing home resident arrives at the clinical microbiology lab of the hospital (time point “A”). Viability assessment such as blood culture (colored bar, yellow for <10 CFU/mL to red for >10⁸ CFU/mL), urine culture (colored bar, green for 10⁴ CFU/mL to red for >10⁵ CFU/mL) or AST culture (colored bar, green for 10⁵ CFU/mL to red for >10⁵ CFU/mL) is the most time-consuming step of all ID or AST procedures as indicated in the diagnostic timeline in FIG. 7 because the shortest phenotypic growth time needed is determined by the ability to detect the most fastidious species on the selected ID panel. Similarly, the total time needed for AST is determined by the slowest-acting antibiotic in the panel. The TAT of the streamlined ID/AST assay directly from clinically relevant specimens such as urine and whole blood with the current system in the blue block in FIG. 7 was optimized based on these criteria. A single culture-positive or culture-negative reporting does not warrant an immediate change of antibiotic therapy, but a timely AST reporting could trigger a switch to alternative antibiotic regimens. However, as illustrated in the “Susceptibility timeline” at the bottom in FIG. 7, the time to AST reporting (“E”) can range from hours to days. For the conventional blood culture method, AST results will not be available in a few days (“E7”) after the specimen arrives at the clinical lab (“A”), and the current systems can report AST within the same day directly from urine (“E5”) or whole blood samples (“E6”). Since the proposed patient-side initiated molecular test starts in the nursing home even before the ambulance or medical transfer vehicle arrives, the AST results could be available 3-6 hours after the specimen transfer module arrives at the clinical lab (E1, E2 and E3) for urine samples or −11 hours after for whole blood samples (E4).

The current pathophysiologic paradigm of septic shock (SIRS, SOFA, MODS, 3-100s or others) fails to appropriately consider the primacy of the microbial burden of infection as a key driver of septic organ dysfunction, because currently there is no technology available to assess the microbial burden at the time of treatment. This view frames sepsis as an immunologic syndrome that is only indirectly related to the underlying infection, and in actuality SIRS can be caused by ischemia, inflammation, trauma, surgery complications, infection or several insults combined. Nearly every ICU patient (sometimes reported greater than 90%) fits the SIRS criteria, but not all SIRS patients are septic. There are subgroups of sepsis patients particularly at extremes of age such as nursing home residents who do not meet criteria for SIRS on presentation but progress to severe infection and multiple organ dysfunction and death. The disclosure looks to enabling a patient-side initiated molecular test to phenotype the microbial infectious load, which substantially drives downstream responses including the development of organ dysfunction and septic shock. Since sepsis is presumed to result from underlying infection, it can be inherently classified by microbial burden with individual-level data and multiple phenotypes (viable/dead, Gram+/Gram−, susceptible/resistant, etc.) specifying the underlying and chain (intermediate or immediate) causes of septic conditions during the transfer event from nursing homes to hospitals with a new viability transfer module.

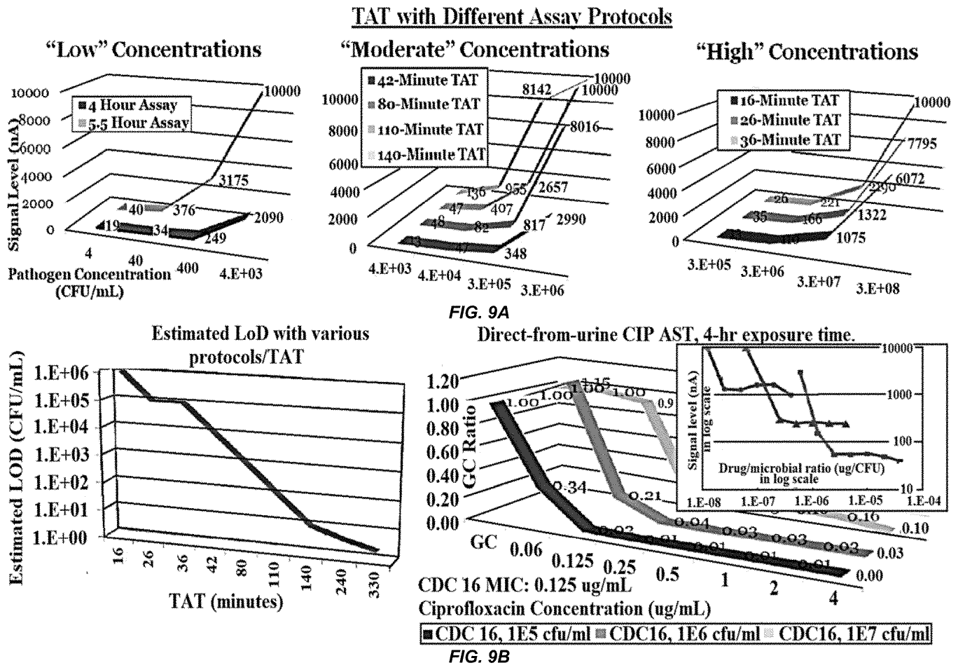

Microbial infectious load classification can be simply stratified as target pathogen concentrations from 0 to >10⁸ CFU/mL in different specimen types along with other phenotypes such as Gram+/Gram−, vancomycin (R/S), carbapenem (R/S) or bacterial (yes or no). The correlation between the limit of detection (LoD) of the current molecular analysis platform (FIG. 7 blue box) with the assay turnaround time (TAT) was established in FIG. 9A-B in order to determine the minimum assay time needed for quantification of RNA transcription at different levels of pathogen concentrations. As shown in FIG. 9A, the TAT and dynamic range of ID can be configured to be from 16 minutes to 36 minutes by adjusting the lysate incubation time for higher target LoDs. Target pathogen enrichment and matrix component removal can be carried out by the built-in centrifugal module to achieve lower target LoDs with TAT of 42 minutes to 140 minutes. For low-abundance pathogens and early infection diagnostics, additional viability assessment steps (colored bar in FIG. 7) with TAT of 4 hours to 5.5 hours can be included to achieve an LoD of <10 CFU/mL. The direct-from-specimen AST protocol will be based on these assay parameters to adjust the antibiotics exposure time (colored bar in FIG. 7) for triple dynamic algorithm as described below.

The research from the initial studies was to streamline the blood pelleting procedure into the integrated system to remove the blood matrix and recover all pathogens from the cellular pellet through a lysis-centrifugation module in the lab automation systems. Whole blood has one of the most complex matrices, and many matrix components can affect the signal response of a bioanalytical process. Blood matrix components such as IgG, hemoglobin and lactoferrin have been described as inhibitors of PCR. Serum proteins often bind nonspecifically to analytes or the sensor surface, resulting in reduced sensitivity. The high viscosity of blood also alters the binding efficiency and specificity for detection.

The incorporation of an embedded blood lysis-centrifugation for bacterial/cellular pelleting can effectively address the blood matrix composition difference from urine and low abundant bacteria in extremely low volume blood samples from critically ill patients. The lysis-centrifugation technique (Isolator tube) was commonly used in evaluating clinically relevant titers of bacteria in sepsis patients even for <1 CFU/mL in the mid-1980s, but was later replaced by automated blood culture systems such as BD BACTEC™ and bioMérieux BacT/ALERT®, which take overnight to days for results. The device and methods seek to separate the sample processing module into a patient-side specimen processing device to initiate viability assessment during transportation. Since the phlebotomist at the nursing home can remove the rubber cap of the specimen tube (whole blood or urine) and insert into the proposed device along with the transport cartridge, most of the sample processing components (tube gripper, tube rack, tube transfer actuator and de-capping module) will not be needed in the patient-side compact device. A microcentrifuge, normally used for hematocrit centrifugation in 2 mL tubes, will be mounted with a special rotor spindle for 4 mL tubes and eventually 10 mL tubes on an electric motor MUB504VF (5000 RPM) and operated by the current closed-loop controller with armature voltage feedback. The centrifugal concentration module will be constructed to International Electrochemical Commission (IEC) standard 61010-2-020.

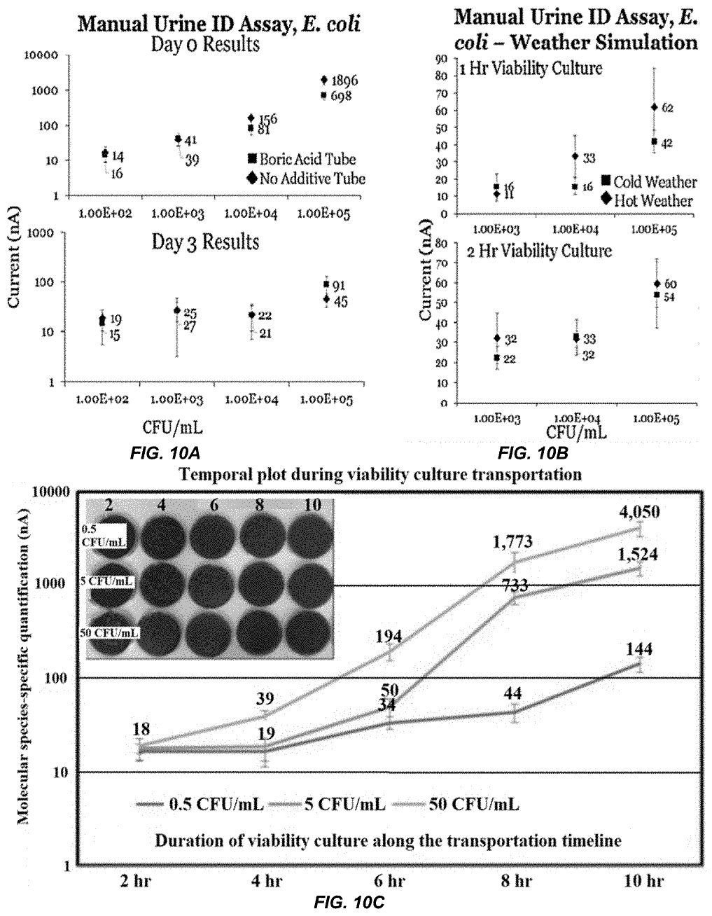

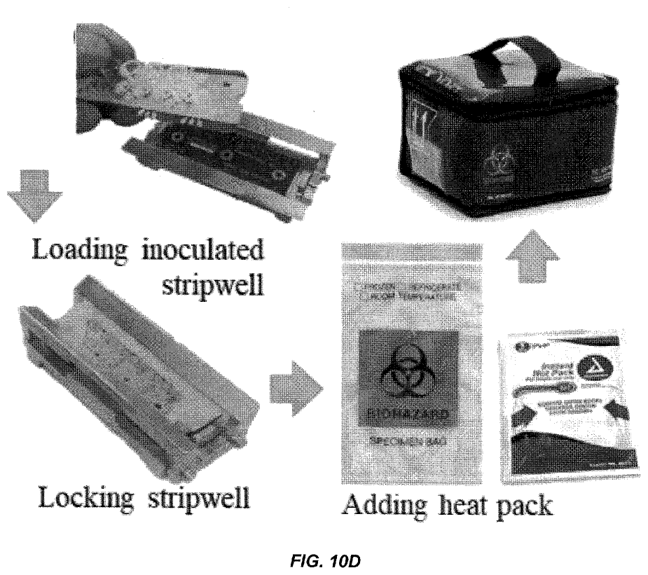

A recent study demonstrated the measurement of 21 blood biomarkers from 134 blood samples taken by emergency medical services (EMS) and placed in a Coleman cooler box on the worktop inside the patient's cabin in an ambulance truck during the hospital transfer was exactly like the one immediately analyzed in the hospital setting. The possible benefits to patient outcome deriving from out-of-hospital blood sampling were limited in this study, because blood samples were taken by EMS, and the time saving is only 30 minutes or less on the ambulance ride to the emergency department in the hospital. All residents of long-term care facilities with suspected symptomatic infection should have appropriate diagnostic tests done promptly early in the process, preferably not right before hospital transfer, and the findings should be discussed with the primary care clinician. There is a need for better tests to rapidly identify patients with resistant pathogens and for more judicious use of broad-spectrum antibiotics for empiric sepsis treatment, especially in nursing home settings. Through the current pre-clinical verification with various transportation options (tube types, weather conditions, duration, boric acid) for direct-from-urine ID/AST on UtiMax, the viable pathogen can be detected with species-specific molecular assays while contaminants (skin flora at concentrations <10⁴ CFU/mL) were not reported positive in FIG. 10A. Just as demonstrated in FIGS. 9A and B, the clinical cutoff (such as 10⁴ CFU/mL for urine) can be adjusted by varying the viability assessment time after being transported with a cold pack or heat pack. Aliquots of whole blood samples contrived with bacteria at concentrations from 0.5, 5 to 50 CFU/mL were processed and placed in the transportation pack and one aliquot per concentration was taken out every 2 hours to quantify the species-specific 16S rRNA for bacterial growth monitoring as shown in FIG. 10C. For whole blood samples, the transportation time from nursing home to the clinical microbiology lab might not be long enough for 0.5 CFU/mL to report positive while both 5 and 50 CFU/mL reported positive after 6 hours in the transportation package. Contrive blood samples at 0.1, 0.5, 1, 5 and 10 CFU/mL will be generated. The patient-side device will equip a tube label printer to print out patient coded info, specimen type and collection time. So the main system in the clinical microbiology lab can scan the info and proceed with molecular quantification if transportation time is longer than the minimum viability assessment time (2 hours for an LoD of 10⁴ CFU/mL with urine and 9 hours for an LoD of 0.5 CFU/mL with whole blood) or continue to incubate the transportation pack until the minimum viability assessment time is up. All contrived concentrations were verified with agar plating according to CLSI guidelines as used in our current clinical protocols.

TABLE 1

| | Time to | | | |
|---|---|---|---|---|
| | arrive at | Collection | | Temperature |
| | the lab | tube | Additive | control |
| EMS | 1 hour (up to | BD 367884 | None | 1 heat pack |
| (Ambulance) | 2 hours) | tube for | | to maintain |
| | | blood, BD | | growth |
| | | 364954 or BD | | |
| Medical | Several hours | 362725 for | With or | 1, 2, or 3 |
| Specimen | to overnight | urine and | without | heat packs |
| Courier | | swab | boric acid | to maintain |
| | | | if urine | growth |
| | | | overnight | |

The disclosure provides a method comprising collecting a patient sample or specimen, aliquoting the specimen into one or more carrier devices (e.g., culture tube, microwell culture devices or strip-well device), optionally packaging the collected specimen with a temperature system and a timer or time-stamp. In one embodiment, the patient sample or specimen is selected from urine, blood, sputum, surgical drain fluids, spinal fluid, or biological swab). In another embodiment, the carrier devices is a multiwall system comprising a plurality of wells wherein each well comprises (i) different antimicrobial agents, (ii) different concentrations of the same antimicrobial agent or (iii) a combination of (i) and (ii). In still another or further embodiment, the temperature system comprises a heating pack or a cold pack or a device that can regulate the temperature. In another embodiment, the sample is a urine sample and the temperature system is a cooling pack or a system that maintains the temperature below 37° C. (e.g., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., 0° C.). In yet another embodiment, the sample is a blood sample and the temperature system is a warm/heating pack or system that maintains the temperature at about body temperature (e.g., about 35-39° C.).

In one embodiment, a stripwell system comprises a plurality of culture wells (e.g., 100-500 μl working volumes) arranged in a strip. The strip well system can comprise a plurality of antibiotics commonly prescribed for complicated urinary tract infections and sepsis (e.g., ciprofloxacin (fluoroquinolone class), gentamicin (aminoglycoside class), and meropenem (carbapenem class)). In some embodiments, the stripwells can be used for AST and one-drug testing. AST stripwells can contain all three antibiotics with various concentrations (e.g., two concentrations for ciprofloxacin and gentamicin and three concentrations for meropenem). One-drug stripwells use only one antibiotic and can include a broader range of concentrations of two-fold dilutions in order to determine the antimicrobial concentration needed to inhibit microbial growth. Additionally, a stripwell configuration can comprise one well without antibiotics to be used as a growth control (GC).

For example, a one-drug stripwell can contain a plurality of concentrations for only one antibiotic. The first well of each stripwell can be left with no antibiotic to be used as a growth control. Ciprofloxacin one-drug stripwells are prepared at the following concentrations: 0.25, 0.5, 1, 2, 4, 8, and 16 μg/mL. Gentamicin one-drug stripwells are prepared at the following concentrations: 0.25, 0.5, 1, 2, 4, 8, and 16 μg/mL. Meropenem one-drug stripwells are prepared at the following concentrations: 0.25, 0.5, 1, 2, 4, 8, and 16 μg/mL.

The AST stripwell can contain a number of antibiotics and includes, for example, the following concentrations for each antibiotic: 1 and 4 μg/mL ciprofloxacin, 2 and 8 μg/mL gentamicin, and 1, 4, and 16 μg/mL meropenem. Antibiotic solutions are added to their corresponding wells. The first well is left with no antibiotic to be used as a growth control during testing.

In one embodiment, the samples are loaded onto stripwells as appropriate for the test to be performed and are packaged according to the type of sample (e.g., cold for urine; warm for blood etc.) and shipped with an optional timer and/or time stamp to determine transit time to a laboratory.

The disclosure also comprises a kit for carrying out the methods of the disclosure. The kit can comprise one or more carrier devices (e.g., culture tube, microwell culture devices or strip-well device), optionally a temperature system and a timer or time-stamp notice. In one embodiment, the kit comprises one or more carriers devices for receiving a patient sample or specimen selected from urine, blood, sputum, surgical drain fluids, spinal fluid, or biological swab). In another embodiment, the carrier devices is a multiwall system comprising a plurality of wells wherein each well comprises (i) different antimicrobial agents, (ii) different concentrations of the same antimicrobial agent or (iii) a combination of (i) and (ii). In still another or further embodiment, the temperature system comprises a heating pack or a cold pack or a device that can regulate the temperature. In another embodiment, the sample is a urine sample and the temperature system is a cooling pack or a system that maintains the temperature below 37° C. or at body temperature. In another embodiment, the microwells or tubes comprise culture media. In any number of the foregoing embodiments, the collection device comprises sterile culture media.

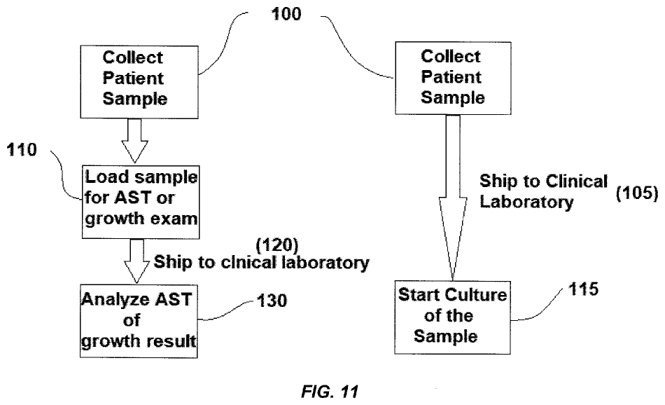

FIG. 11 provides a simple depiction of the advantages of the disclosure compared to current processes. As discussed above, a patient sample is collected (100). The patient sample (100) can be a biological fluid (e.g., urine, blood, sputum, surgical discharge and the like). In current process, this sample is then shipped (105) to a clinical laboratory, which upon receipt begins culture of the sample for microbial growth and AST analysis (115). In this process the time from collection to the start of culture can range from several hours to a day particularly wherein the sample is collected at the bedside in a nursing facility or at home. In a method of the disclosure, the patient sample (100) is immediately loaded into an assay system (e.g., growth medium, with controls or AST system with control) (110) at which time the culture begins prior to shipping (120). In this method upon receipt at the clinical laboratory the growth/AST results (130) can be determined shortly after arrival at the laboratory.

The following aspects of the invention are provided:

Aspect 1: A method to collect, pack and transport the specimens for microbiological testing, which comprises the steps of:
(a) The microbiological testing is determined partially by the change of growth marker to assess microbial burden, antimicrobial susceptibility from identified or unidentified pathogen under various antimicrobial exposure conditions compared to a Growth Control (GC) without any antimicrobials.
(b) The microbial burden is determined partially by the change of growth marker from unidentified pathogen growth within a pre-determined viability culture time as part of the specimen transportation time.
(c) The antimicrobial susceptibility is determined partially by the change of growth marker within the antibiotic exposure time as part of the specimen transportation time from unidentified pathogen diluted to different concentrations with various drug/bug ratios compared to a Growth Control (GC) condition without any antimicrobials.
(d) The antimicrobial susceptibility is determined partially by the change of growth marker from identified pathogen with various drug/bug ratios compared to Growth Control (GC) without any antimicrobials.
(e) The microbial burden and antimicrobial susceptibility are determined by pathogen growth within a pre-determined viability culture time after removing the matrix interference components.
(f) The microbial burden and antimicrobial susceptibility are determined by pathogen growth within a pre-determined viability culture time after concentrating the pathogens in the raw specimens.

Aspect 2: A method as recited in aspect 1, wherein said growth marker includes but is not limited to nucleic acids, proteins, phenotypic characteristics, and visual observation.

Aspect 3: A method as recited in aspect 2, wherein said growth marker is RNA and the change of RNA content is quantified with molecular analysis assays.

Aspect 4: A method as recited in aspect 3, wherein said molecular analysis assays include but are not limited to species-specific quantification, group-specific quantification, universal quantification. Examples of species are E. coli, Klebsiella pneumoniae, and methicillin-resistant Staphylococcus aureus (MRSA). Examples of groups are Enterobacteriaceae, Gram-negative and Gram-positive bacteria.

Aspect 5: A method as recited in aspect 4, wherein said growth marker is RNA and the change of RNA content is quantified with molecular analysis assays with enzymatic signal amplification with electrochemical sensors.

Aspect 6: A method as recited in aspect 1, wherein said pathogen growth includes but is not limited to growth conditions in microdilution, macrodilution, agar plating, growth media culture, and growth in clinical specimens or processed specimens.

Aspect 7: A method as recited in aspect 6, wherein said growth conditions include but are not limited to temperature control, preservatives, breakage prevention, leakage prevention, and differential viability culture time to distinguish contaminants.

Aspect 8: A method as recited in aspect 7, wherein said differential viability culture time to distinguish contaminants include but is not limited to a culture time needed at around the limit of detection.

Aspect 9: A method as recited in aspect 1, wherein said antimicrobial exposure includes but is not limited to microdilution, macrodilution, agar plating, growth media culture, and growth in clinical specimens or processed specimens with antimicrobial conditions.

Aspect 10: A method as recited in aspect 9, wherein said antimicrobial conditions include but are not limited to a set number of antimicrobial concentrations, a range of antimicrobial concentrations, various drug-to-bug ratios, and different antimicrobial exposure times.

Aspect 11: A method as recited in aspect 10, wherein said set number of antimicrobial concentrations includes but is not limited to susceptible, intermediate and/or resistant breakpoints.

Aspect 12: A method as recited in aspect 10, wherein said set number of antimicrobial concentrations includes but is not limited to 2-fold increase or decrease from the susceptible, intermediate and/or resistant breakpoints.

Aspect 13: A method as recited in aspect 10, wherein said set number of antimicrobial concentrations includes but is not limited to more than 2-fold increases or decreases from the susceptible, intermediate and/or resistant breakpoints.

Aspect 14: A method as recited in aspect 10, wherein said set number of antimicrobial concentrations includes but is not limited to 2-12 antimicrobial conditions.

Aspect 15: A method as recited in aspect 1, wherein said different concentrations include but are not limited to a set number of dilution levels from the raw specimen, a range of dilution levels from the raw specimen, and different levels of pathogen concentrating step.

Aspect 16: A method as recited in aspect 15, wherein said different concentrations including but not limited to 1×, 0.5×, 0.3×, 0.1×, 0.01×0.001×0.0001× and/or 0.00001×.

Aspect 17: A method as recited in aspect 1, wherein said removal the matric interference components include but is not limited to the removal of supernatant after the optional centrifugation step.

Aspect 18: A method as recited in aspect 17, wherein said optional centrifugation step includes but is not limited to specimen pre-conditioning such as red blood cell lysis or thinning agent to reduce viscosity.

Aspect 19: A method as recited in aspect 17, wherein said centrifugation step includes but is not limited to centrifugation time (5 min, 10 min, 20 min, etc.), centrifugation force (0.1 G, 0.5 G, 1 G, 2 G, etc.) and/or pellet volume (100 μL, 150 μL, 200 μL, etc.).

Aspect 20: A method as recited in aspect 7, wherein said temperature control includes but is not limited to adding one or several cold packs, the addition of one or several heat packs, the addition of temperature-controlled module, or the addition of a thermal isolated module.

Aspect 21: A method as recited in aspect 7, wherein said preservatives include but are not limited to boric acid.

Aspect 22: A method as recited in aspect 7, wherein said breakage prevention include but are not limited to the addition of an outer packaging box made of sturdy material for protection, such as plastic, wood, or metal.

Aspect 22: A method as recited in aspect 7, wherein said leakage prevention includes but is not limited to the addition of absorbent material, the addition of a sealing bag, and the addition of a sealing tape to each specimen collection tube or container.

Aspect 22: A method as recited in aspect 7, wherein said differential viability culture includes but is not limited to a set viability culture time with preservative and temperature control, a set viability culture time with temperature control but without preservative, and any combination of the use of culture time, temperature control and preservatives.

Aspect 23: A method as recited in aspect 1, wherein said pre-determined viability culture time as part of specimen transportation time includes but is not limited to 5 min., 30 min., 1-hour, 2-hour, 3-hour, 6-hour, 12-hour and 18-hour.

Aspect 24: A method as recited in aspect 1, wherein said various antimicrobials include but are not limited to one, three, five, or ten antimicrobials. Examples of antimicrobials include but are not limited to ciprofloxacin, gentamicin, and meropenem.

Aspect 25: A method as recited in aspect 1, wherein said antimicrobial susceptibility testing includes but is not limited to the susceptibility of a pathogen in a monomicrobial specimen, the susceptibility of pathogens in a polymicrobial specimen, the susceptibility of a multiple-drug resistant pathogen in a monomicrobial infection, and the susceptibility of each multiple-drug-resistant pathogen in a polymicrobial infection

Aspect 26: A method as recited in aspect 10, wherein said set number of antimicrobial concentrations includes but is not limited to 13-96, 96-256, 256-1024 singular antimicrobial conditions to assess antimicrobial susceptibility profiles including but not limited to the susceptibility of each pathogen in a polymicrobial specimen, the susceptibility of a multiple-drug-resistant pathogen in a monomicrobial infection, and the susceptibility of each multiple-drug-resistant pathogen in a polymicrobial specimen.

Aspect 27: A method as recited in aspect 10, wherein said set number of antimicrobial concentrations includes but is not limited to 13-96, 96-256, 256-1024 singular and/or combinational antimicrobial conditions to assess antimicrobial susceptibility profiles including but not limited to the susceptibility of pathogens in a polymicrobial specimen, the susceptibility of a multiple-drug resistant pathogen in a monomicrobial infection, and the susceptibility of each multiple-drug-resistant pathogen in a polymicrobial infection. Combinational antimicrobial conditions include but are not limited to combinations of 2, 3, 4, 5, and 10 antimicrobials in one testing condition.

Aspect 28: A method as recited in aspect 27, wherein said antimicrobial susceptibility profile includes but is not limited to no observed growth, limited growth, minimum growth, and relatively low growth.

Aspect 29: A method as recited in aspect 1, wherein said antimicrobial susceptibility profile includes but is not limited to homogeneous microbial populations, heterogeneous microbial populations, pseudo-homogeneous microbial populations and pseudo-heterogeneous microbial populations.

Aspect 30: A method as recited in aspect 29, wherein said antimicrobial susceptibility profile includes but is not limited to homogeneous resistant microbial population, heterogeneous resistant microbial population, pseudo-homogeneous resistant microbial population and pseudo-heterogeneous resistant microbial population.

Aspect 31: A method as recited in aspect 29, wherein said antimicrobial susceptibility profile includes but is not limited to the case where the majority of a heterogeneous microbial population is susceptible to a given antibiotic and a minority of the population is resistant, so the growth of the minority population will only be observed after the inhibited growth of the susceptible majority is observed.

Aspect 32: A method as recited in aspect 1, wherein said pre-determined viability culture time includes but is not limited to 2-hours, 3-hours, 6-hours, 12-hours and 18-hours in order to observe the growth of a minority population after the inhibited growth of a majority susceptible population.

### EXAMPLES

Bacterial strains and assay materials—Contrived samples were prepared with E. coli ATCC 25922. Sensor chips used for the molecular detection of bacteria were produced in-house using a previously established protocol.

General shipping conditions—Separate packaging conditions for urine and blood samples were optimized to comply with FedEx clinical shipping guidelines, which requires four layers of material: (1) primary watertight inner receptacle, (2) absorbent material, (3) secondary watertight inner receptacle, and (4) sturdy outer packaging. Urine samples were prepared in BD C&S preservative tubes (BD364951; Becton, Dickinson and Company, Franklin Lakes, N.J.). Blood samples were prepared in tubes with no additive (BD366703) with specimen collected in tubes containing lithium heparin (BD367880). Tubes were then taped shut for an additional seal, wrapped in an absorbent material such as paper towel or cloth padding, and placed inside a sealed plastic bag. For urine samples, this bag was placed inside a plastic box with one cold pack. For blood samples, this bag was placed in an additional thermal bag with two heat packs wrapped in bubble wrap, which was then placed inside the outer plastic box. A traceable thermometer was also included in each package to track the temperature profile of shipped specimens.

Evaluation of urine shipping conditions—The initial effort to evaluate urine storage conditions for microbiological cultures was to compare the detection sensitivity of contrived urine samples at concentrations below and above the clinical cutoff of 10⁴ CFU/mL to mimic skin flora contaminations and uropathogens, respectively. Because transportation times may vary depending on distance and other unpredictable factors, the longest possible time, or worst possible scenario, of 3 days was used to simulate a shipment taking place over the weekend using various shipping and assay conditions. To first assess the effects of storage temperature, multiple sets of 4-mL urine samples were prepared in boric acid tubes, each containing two samples spiked with 1×10³ and 1×10⁵ CFU/mL E. coli, and tested them on Day 0 and Day 3 using three conditions. The two sets of samples tested on Day 0 did not undergo any storage conditions as they were tested immediately after sample preparation with either one or two hours of viability culture. In the first condition, one set of samples was stored at 4° C. for three days and allowed one hour to return to room temperature just before testing with a one-hour viability culture. In the second condition, two sets of samples were stored at 4° C. and tested with either one or two hours of viability culture immediately at the end of the three-day period, with no additional hour to return to room temperature prior to testing. The third condition was similar to the second condition with the exception of room temperature storage rather than 4° C. storage.

As most clinical specimens are transported in 24 hours or less, evaluation of the detection sensitivity throughout this more clinically relevant time period was performed, rather than the 3-day period used in the previous experiment, by testing at 0, 12, and 24 hours (lower and upper limits of overnight shipping time). Urine samples were prepared with E. coli at 1×10², 1×10³, 1×10⁴, and 1×10⁵ CFU/mL. Two shipping and assay conditions were tested during this experiment. One condition included samples prepared in boric acid tubes and packaged with one cold pack that were tested with either 1 or 2 hours of viability culture. The second condition was designed to evaluate the use of tubes without any additive and with one heat pack, essentially taking no additional measures to preserve the bacterial sample. Samples were then tested at 0, 12, and 24 hours with a manual ID assay.

After various efforts to optimize shipping conditions, an enhanced shipping protocol was tested by simulating an overnight shipment in-house. Two sets of urine samples were prepared per condition in boric acid tubes, with each set including one negative urine sample (“blank”) and samples spiked with E. coli at 5.5×10², 5.5×10³, and 5.5×10⁴ CFU/mL. The first set (Day 0, before shipping) was tested immediately after preparation with 1 and 2 hours of viability culture. The second set (Day 1, after shipping) was packaged with one cold pack according to the optimized shipping protocol and tested the following day after the “overnight shipping” period, or the 19 hours from sample preparation to an 8 AM morning delivery, with a 1 and 2-hour viability culture.

Evaluation of blood shipping conditions—The main consideration for the design of the blood transportation pack was to enhance the recovery rate of viable bacteria, especially for an extremely low colony count (<1 CFU/mL). Because lower colony counts require longer culture times to reach the limit of detection, the recovery rate of varying concentrations of bacteria in blood was assessed by testing 2-mL samples prepared in no additive tubes with E. coli at concentrations of 0.47, 4.7, and 47 CFU/mL. All samples were packaged in thermal bags with 2 heat packs and tested every two hours for ten hours with the blood ID assay. The contrived density was verified by blood agar plating.

The described conditions were then assessed under transportation times longer than the 10 hours tested previously and observe if this longer transportation would lead to overgrowth of bacteria and affect detection sensitivity. Additionally, we wanted to further optimize the incubation conditions during transportation by testing the number of heat packs. To simulate overnight shipping, blood samples spiked with E. coli at 0.83 and 5.3 CFU/mL with a 2-mL starting volume were prepared. Samples underwent red blood cell lysing, followed by resuspension in Mueller-Hinton II (MH) broth, and were packaged with either one or two heat packs to simulate the overnight transportation time of 15-20 hours before testing. This incubation period served to replace the viability culture portion of our blood ID assay. Samples were then tested with the blood ID assay upon receipt.

Assessment of feasibility of transportation protocols—To test the improved preparation and shipping protocols for both urine and blood specimens, New York-Presbyterian Queens Hospital (NYPQ) was asked to contrive and ship urine and blood samples using optimized conditions. Urine samples were prepared in C&S tubes at 1×10³, 1×10⁴, and 1×10⁵ CFU/mL E. coli and packaged with one cold pack. Since there is a clinical cutoff for urine pathogen ID, a cold pack and boric acid were used to inhibit the growth during transportation. Blood samples were prepared in tubes containing no additive at 1 and 5 CFU/mL E. coli and packaged with no heat packs due to a shortage at the time of testing. Samples were packaged according to FedEx guidelines and shipped overnight to GeneFluidics. After approximately 22 hours from the time of sample preparation to the time of delivery, all samples were tested with their respective ID assays.

ID assay procedure—Urine samples of 4-mL starting volume were spun in a centrifuge at 5,000 RPM for 5 minutes, after which supernatant was removed and replaced with cation-adjusted MH broth. Samples were then cultured according to the conditions of the experiment. After the viability culture, the samples underwent a second round of centrifugation and supernatant removal, leaving 150 uL of sample. The samples were then lysed by adding 1M NaOH with a 5-minute incubation at room temperature, followed by the addition of 1M HCl. Lysate was then delivered to all sensors on the sensor chip, with no sample being delivered to the negative control sensor. The chip was incubated for 30 minutes at 43° C., then washed with distilled water to remove non-specific binding and dried with pressurized air. Horseradish peroxidase was delivered to every sensor on the chip and incubated for five minutes before a second wash and dry cycle. TMB was subsequently added to each sensor on the chip. After a 30-second incubation, the electrochemical signal generated by the chemical reaction of the HRP with the TMB and the applied voltage across the gold electrodes was measured by a 16-channel potentiostat reader.

Blood samples starting at 2 mL underwent a similar assay procedure with the exception of two additional rounds of red blood cell lysing before the addition of MH broth. Prior to packaging and culturing in MH broth, samples were lysed two times with 100 mg/dL saponin and incubated at room temperature for 10 minutes, followed by 5 minutes of centrifugation. Blood samples did not undergo a viability culture during the assay, as they were cultured during transportation according to the conditions of each experiment.

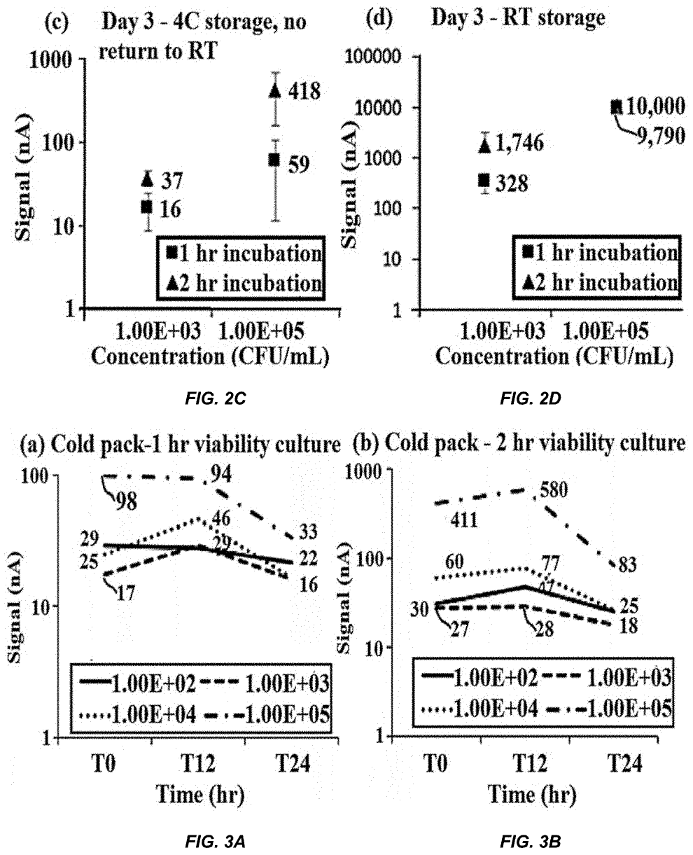

Urine storage conditions to maintain detection sensitivity. The desirable conditions should report both contamination and uropathogens after storage and transportation the same way as tested immediately at T=0 (FIG. 2a). As seen in the test summary in Table 1, urine stored at 4° C. without an additional hour to return back to RT before testing (FIG. 2c) resulted in pathogen detection reporting and signal levels similar to those generated during immediate testing (FIG. 2a). Samples of 10³ CFU/mL, which are above the clinical cutoff of 10⁴ CFU/mL, remained positive; samples of 10³ CFU/mL, which were reflective of skin flora contamination, remained negative. Much higher signal levels for both concentrations, but more importantly for 10³ CFU/mL samples, were observed in FIGS. 2b and 2d, indicating bacterial overgrowth, which could result in false positives caused by overgrown contaminants.

TABLE 1
Test conditions and pathogen detection
reulst summary from FIG. 2A-D.

| | Contrived urine | Contrived urine |
|---|---|---|
| Conditions | at 10³ CFU/mL | at 10⁵ CFU/mL |
| Control set | Not detected with | Reported positive with |
| tested | either 1 hr or 2 hr | both 1 hr and 2 hr |
| immediately | viability culture | viability culture |
| at T = 0 | | |
| Store at 4° C. | Not detected with | Reported positive with |
| with 1 hr | 1 hr viability | 1 hr viability culture |
| return to | culture | with exceedingly high |
| RT (1b) | | signal indicating |
| | | overgrowth. |
| Store at 4° C. | Not detected with | Reported positive with |
| without | either 1 hr or 2 hr | both 1 hr and 2 hr |
| return to | viability culture | viability culture |
| RT (1c) | | |
| Store at RT (1d) | Not detected with | Reported positive with |
| | 1 hr viability | both 1 hr and 2 hr |
| | culture, but | viability culture with |
| | reported positive | exceedingly high |
| | with 2 hr | signal indicating |
| | viability culture | overgrowth |

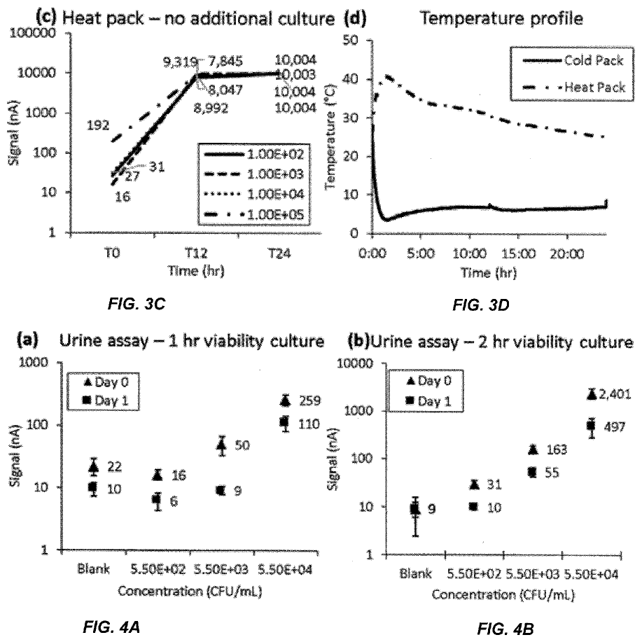

In a follow-up experiment to test a more clinically relevant timeframe, it was found that for urine samples packaged with one cold pack and tested with only one hour of viability culture, those at or below clinical cutoff (10², 10³, 10⁴ CFU/mL) were reported negative while urine samples contrived at 10³ CFU/mL were reported positive after 12 hours of transportation. However, after 24 hours of transportation the 10⁵ CFU/mL sample was reported negative, indicating that a longer viability culture is needed to bring the pathogen out from the stationary phase. With a 2-hour viability culture, as shown in FIG. 3b, the 10⁵ CFU/mL sample was reported positive after 12 and 24 hours of transportation. For this condition, signal levels from T12 (12-hour transportation) were comparable to those from TO (tested immediately). If the specimen is transported through a commercial carrier such as FedEx, the temperature inside the delivery truck could be highly elevated as simulated with a heat pack in FIG. 3c, causing overgrowth of all contrived conditions. FIG. 3d shows the thermal profile recorded with a traceable thermometer inside the transportation pack with cold or heat packs.

Simulated urine specimen transportation. Results for a simulation of overnight shipping of urine specimens showed that detection sensitivity was comparable if the pathogen ID assay included a 2-hr viability culture. The urine sample spiked at 5.5×10³ CFU/mL could not be detected after FedEx Clinical Pak shipping if the viability culture was only one hour as shown in FIG. 4.

Blood specimen transportation pack to enhance viable recovery rate. Results from an effort to enhance the viable recovery rate of blood samples confirmed that low colony samples would require a longer viability culture time to reach the limit of detection to be reported positive. In FIG. 5a, contrived blood samples reported positive after 6 hours for 4.7 and 47 CFU/mL and 10 hours for 0.47 CFU/mL, as expected. Table 2 shows the blood agar plating to verify the contrived concentration. In a simulated overnight blood specimen transportation via FedEx Clinical Pak shipment with contrived whole blood at 0.83 and 5.3 CFU/mL, two heat packs were needed to report both concentrations positive as shown in FIG. 5b. All four samples spiked at 5.3 CFU/mL produced positive results with one or two heat packs, although those packaged with one heat pack generated a lower signal level than those packaged with two heat packs. For the set of samples spiked at 0.83 CFU/mL, there was at least one sample for each heat pack condition that produced a negative result, as expected due to the probability of one colony existing in the 2-mL blood volume at 0.83 CFU/mL.

TABLE 2
Colony count at each time point of testing for FIG. 5a:

| Concentration | | 2 hours | 4 hours | 6 hours | 8 hours | 10 hours |
|---|---|---|---|---|---|---|
| 0.47 | CFU/mL | 0 | 65 | 500 | Too many | Too many |
| | | | | | to count | to count |
| 4.7 | CFU/mL | 11 | 199 | Too many | Too many | Too many |
| | | | | to count | to count | to count |
| 47 | CFU/mL | 164 | 500 | Too many | Too many | Too many |
| | | | | to count | to count | to count |

An additional experiment in which NYPQ prepared and shipped blood and urine specimens overnight to GeneFluidics demonstrated the feasibility of the improved specimen transportation protocols for both blood and urine. As shown in FIG. 6a, all six contrived blood samples (three at 1 CFU/mL and three at 5 CFU/mL E. coli) were reported positive after approximately 22 hours between sample preparation and testing following transportation. In FIG. 6b, only the E. coli urine sample contrived at 10⁵ CFU/mL and cultured for 2 hours during the assay tested positive, and samples of concentrations reflective of potential skin flora contaminations (<10⁵ CFU/mL) were not reported positive as designed.

To assess the feasibility of direct-from-specimen AST, contrived urine and blood samples were used at two different concentration levels to simulate high and low levels of microbial infectious load. Semi-automatic AST tests directly from whole blood and urine contrived samples were evaluated. Additional mechanical and programming tasks are implemented in order to conduct fully automated direct-from-specimen AST tests with multiple specimen types. With an antibiotic exposure of 4 hours for blood and 2 hours for urine, resistant strains (E. coli CDC 55) can be differentiated from susceptible strains (E. coli CDC 77). To explore biological, chemical and molecular analytical limitations, shorter antibiotic exposure times of 30 and 90 minutes were used to assess the separation of responses curves from both resistant and susceptible strains.

Whole blood samples in BD 367884 Vacutainer Lithium Heparin tubes or urine/swab samples in BD 364954 Vacutainer® Plus C&S Preservative Tubes can be directly loaded into the patient-side specimen processing device and the bacterial pellet can be inoculated at two different concentrations into the AST stripwells. Since there are no commercially available FDA-cleared systems or CLSI reference methods to provide AST results directly from specimens without overnight culture or clinical isolates, the acceptance criteria for the analytical validation and clinical testing will be the same as conventional AST to demonstrate >95% categorical agreement on 150 contrived specimens (50 blood, 50 urine, and 50 swab) both at GeneFluidics (GF) and NYPQ.

Analytical validation protocol: (1) The user loads the specimen collection tube into the device, which then (2) scans the bar code to determine specimen type. (3) Image recognition function determines the volume of the specimen. (4) The system pellets the sample by spinning down then removing supernatant once for urine and swab samples and twice for blood. (5) The system inoculates the pellet with culture media into 1× inoculum, then (6) aliquots and dilutes into two additional inoculum concentrations with dilution factors. (7) The 3 inoculums are added into 3 stripwells for antibiotic exposure inside the system and the remaining suspension can be plated or added to glycerol for archival or retesting. (8) The samples are lysed then delivered to sensors for hybridization, followed by (9) stringency wash. The system incubates enzyme, then performs electrochemical reading and (10) AST reporting.

A simple method using an absolute cutoff is generally sufficient to achieve essential agreement (EA) for most samples, but there are certain more complicated scenarios that require a more complex algorithm to determine a MIC value. Direct-from-specimen AST is particularly demanding because the starting pathogen concentration is unknown and can range from 10³-10⁸ CFU/mL for clinical urine samples and <1-10³ CFU/mL for clinical whole blood samples; conventional methods of generating MIC values from AST usually require a precise bacteria concentration of 5×10⁵ CFU/mL. Variations in this starting concentration have been shown to have a significant effect on the observed MIC value, especially for carbapenems such as meropenem. In the current categorical reporting AST, inoculum concentrations near 10⁷ CFU/mL are predetermined and generated by the system in order to match the microbiological response within 2 hours instead of the conventional antibiotic incubation time. To address the issue of uncertainty (unknown species, unknown microbial load, unknown susceptibility), BsiMax utilizes a triple-kinetics AST that tests susceptibility based on GC response plus two initial pathogen concentrations: the original pathogen concentration present in the sample (if present), and a second concentration diluted tenfold prior to AST culture. The first kinetic curve is from two growth control (GC) wells with differential culture medium for Gram + and Gram − and different lysing reagents are used to release the nucleic acid content after the antibiotic exposure. This viability response is critical to determine the correct drug-to-microbial ratio for susceptibility reporting. The second and third kinetic curves are microbial response to antibiotic conditions. Initial data demonstrates a wide range of drug-to-microbial ratios (μg/mL of antibiotic divided by the microbial concentration in CFU/mL) with significant overlap between each set of microbial response curves for each of three tenfold inoculum concentrations. Any two sets of kinetic curves can cover three orders of magnitude of drug/microbial ratios. This range of concentrations, even if they do not capture the typical 5×10⁵ CFU/mL concentration used in most AST evaluations, provides more information than one dataset alone and allows the BsiMax algorithm to consider the concentration effects of samples with either very high concentrations or concentrations near the LoD.

The current electrochemical-based biosensor measures the reduction current from cyclic enzymatic amplification of a horseradish peroxidase (HRP) enzyme label with TMB and H₂O₂. The resulting current signal can be estimated with the Cottrell equation. Each triple-response-curve signature will be generated by overlaying GC response with two curves of all drug/microbial conditions with identified trends in GC ratios while increasing antibiotic concentrations, establishing a signature library corresponding to each inoculum concentration. Changes in response signature will be analyzed by the current algorithm with a trend of categorical classification change (such as always susceptible, ranging from susceptible to resistant or always resistant), prior to the contrived blood analytical validation study. Contrived bacterial species will be crosschecked by finding the closest matches in GC ratio trending of triple-response-curve signatures of each unknown bacteria to the ones in the signature library. The categorical classification of hundreds of different reference targets, pathogens without susceptibility profiles or even unknown pathogens can theoretically be identified and distinguished with the same triple-response-curve signature in this manner.

In an LoD verification experiment based on FDA guidelines, all 24 whole blood samples of 500 mL spiked at 6 CFU/mL tested positive. The spiked concentration was verified by plating 1 mL of the original sample on blood agar. The TAT is 6.5 hours, and the LoD is confirmed to be 6 CFU/mL with all positive signals well above the limit of blank (LOB). The LoD will be further improved by taking larger blood volumes (2-8 mL) and utilizing a longer viability incubation time.

What is claimed is:

1. A method to collect, pack and transport a specimens for microbiological testing, which comprises:
obtaining a specimen for microbiological testing;
inoculating the specimen in a plurality of wells, wherein each well comprises different dilutions and/or different antimicrobial agents;
determining a change of one or more growth markers to assess a microbial burden or an antimicrobial susceptibility from an identified or an unidentified pathogen(s) under various antimicrobial exposure conditions compared to a Growth Control (GC) condition without any antimicrobials,
wherein the microbial burden is determined by a change of the one or more growth markers from unidentified pathogen growth within a pre-determined viability culture time as part of the specimen transportation time; and/or
wherein the antimicrobial susceptibility is determined by a change of one or more growth markers within an antibiotic exposure time as part of the specimen transportation time from unidentified pathogen diluted at different dilution levels with various drug and/or pathogen ratios compared to a Growth Control (GC) condition without any antimicrobials and/or
wherein the antimicrobial susceptibility is determined by a change of one or more growth marker from identified pathogen with various drug and/or pathogen ratios compared to Growth Control (GC) without any antimicrobials, and/or
wherein the microbial burden or antimicrobial susceptibility are determined by pathogen growth within a pre-determined viability culture time after removing a matrix interference components, and/or
wherein the microbial burden or antimicrobial susceptibility are determined by pathogen growth within a pre-determined viability culture time after concentrating the pathogens in the raw specimens.

2. The method of claim 1, wherein the one or more growth markers comprises nucleic acids, proteins, phenotypic characteristics, and/or visual observation.

3. The method of claim 2, wherein the one or more growth markers is RNA and the change of RNA content is quantified with a molecular analysis assay.

4. The method of claim 3, wherein the molecular analysis assay is selected from the group consisting of species-specific quantification, group-specific quantification, and universal quantification.

5. The method of claim 1, wherein the identified or unidentified pathogen is selected from the group consisting of E. coli, Klebsiella pneumoniae, and methicillin-resistant Staphylococcus aureus (MRSA).

6. The method of claim 1, wherein the identified or unidentified pathogen are selected from Enterobacteriaceae, Gram-negative and Gram-positive bacteria.

7. The method of claim 4, wherein said growth marker is RNA and the change of RNA content is quantified with molecular analysis assays with enzymatic signal amplification with electrochemical sensors.

8. The method of claim 1, wherein microbial growth comprises a growth condition in microdilution, macrodilution, agar plating, growth media culture, growth in clinical specimens or processed specimens.

9. The method of claim 8, wherein the growth conditions comprise temperature control, a preservative, breakage prevention, leakage prevention, and differential viability culture time to distinguish contaminants.

10. The method of claim 8, wherein differential viability culture time to distinguish contaminants comprises a culture time needed at around the limit of detection.

11. The method of claim 1, wherein said antimicrobial exposure conditions comprise microdilution, macrodilution, agar plating, growth media culture, or growth in clinical specimens or processed specimens with antimicrobial conditions.

12. The method of claim 11, wherein antimicrobial conditions comprise a set number of antimicrobial concentrations, a range of antimicrobial concentrations, various drug-to-microbe ratios, and/or different antimicrobial exposure times.

13. The method of claim 12, wherein the set number of antimicrobial concentrations is comprised of susceptible, intermediate and/or resistant breakpoints.

14. The method of claim 12, wherein the set number of antimicrobial concentrations is comprised of at least 2-fold increase or decrease from a susceptible, intermediate and/or resistant breakpoints.

15. The method of claim 12, wherein the set number of antimicrobial concentrations comprises 2 to 12 antimicrobial conditions.

16. The method of claim 1, wherein said different dilution levels are comprised of a set number of dilution levels from a raw specimen, a range of dilution levels from a raw specimen, and different levels of pathogen concentrating step.

17. The method of claim 16, wherein said different dilution levels are selected from 1×, 0.5×, 0.3×, 0.1×, 0.01×0.001×0.0001× and/or 0.00001×.

18. The method of claim 1, further comprising removal of supernatant from the specimen and before determining the microbial burden or antimicrobial susceptibility after a centrifugation step.

19. The method of claim 18, wherein said centrifugation step can include a specimen pre-conditioning step comprised of red blood cell lysis or thinning agent to reduce viscosity.

20. The method of claim 9, wherein temperature control is accomplished by using one or more cold packs, one or more heat packs, use of a temperature-controlled device, or use of a thermal isolated device.

21. The method of claim 9, wherein said preservative comprises boric acid or a composition that inhibits growth of a pathogen or cells.

22. The method of claim 9, wherein differential viability culture comprises a set viability culture time with preservative and temperature control, a set viability culture time with temperature control but without preservative, and any combination of the use of culture time, temperature control and preservatives.

23. The method of claim 1, wherein said pre-determined viability culture time as part of specimen transportation time is selected from 5 min, 30 min, 1-hour, 2-hours, 3-hours, 6-hours, 12-hours, 18-hours and 24-hours.

24. The method as recited in claim 1, wherein said various antimicrobials comprise one, three, five, or ten or more antimicrobials.

25. The method of claim 1, wherein said antimicrobial susceptibility testing comprises the susceptibility of a pathogen in a monomicrobial specimen, the susceptibility of pathogens in a polymicrobial specimen, the susceptibility of a multiple-drug resistant pathogen in a monomicrobial infection, and the susceptibility of each multiple-drug-resistant pathogen in a polymicrobial infection.

26. The method of claim 12, wherein said set number of antimicrobial concentrations comprises 13-96, 96-256, 256-1024 singular and/or combinational antimicrobial conditions to assess antimicrobial susceptibility profiles comprising the susceptibility of each pathogen in a polymicrobial specimen, the susceptibility of a multiple-drug-resistant pathogen in a monomicrobial infection, and the susceptibility of each multiple-drug-resistant pathogen in a polymicrobial specimen.

27. The method of claim 26, wherein said antimicrobial susceptibility profile comprise no observed growth, limited growth, minimum growth, and/or relatively low growth.

28. The method of claim 1, wherein said antimicrobial susceptibility profile is performed on a homogeneous microbial population, a heterogeneous microbial population, a pseudo-homogeneous microbial population and/or a pseudo-heterogeneous microbial population.

29. The method of claim 1, wherein said pre-determined viability culture time comprises 2-hours, 3-hours, 6-hours, 12-hours and 18-hours in order to observe the growth of a minority population after the inhibited growth of a majority susceptible population.

30. The method of claim 8, wherein clinical specimens is a swab or a bodily fluid selected from the group consisting of urine, blood, sputum, or surgical drain fluids.
